(19) United States
(12) Patent Application Publication
(10) Pub. No.: US 2002/0055120 A1
(43) Pub. Date: May 9, 2002

(54) DEVICE AND PROCESS FOR THE DETERMINATION AND/OR REMOVAL OF A SUBSTANCE IN A SAMPLE

(76) Inventor: BERND PEVEC, CASTROP-RAUXEL (DE)

Correspondence Address: COLLARD & ROE, 1077 NORTHERN BOULEVARD, ROSLYN, NY, 11576

(21) Appl. No.: 09/101,665

(22) PCT Filed: Jan. 15, 1997
(86) PCT No.: PCT/EP97/00153

(30) Foreign Application Priority Data
Jan. 16, 1996 (DE) 196 01 346.1

(51) Int. Cl. G01N 33/53

(52) U.S. Cl. 435/7.1

(57) ABSTRACT

A method for the determination and/or removal of a substance in or from a sample by reacting said substance with complexing agents, characterized in that

said substance has at least two domains which will interact with complexing agents;

complexing agents are used which have at least two positions which will interact with said at least two domains;

the complexing agents are added to the sample, and after a complex formation time;

the sample is sorted by the size of the formed complexes by means of a separating device sorting by size in which the formed complexes are retained and can be detected.

The present invention relates to a method for the determination and/or removal of a substance in or from a sample by reacting said substance with complexing agents, and to a device for performing said method.

Methods of the generic type are, in particular, those based on affinity reactions wherein the substances to be determined are labeled by reaction with affinity ligands, for example, antibodies, and then detected through a secondary reaction. Such reactions are rather complicated in part, and prone to interferences. In addition, they require a high instrumental expenditure. Thus, the object of the present invention has been, inter alia, to provide a method which is faster and simpler and yet provides equally reliable results about the presence of a substance in a sample to be examined.

This object is achieved by a method with the features of claim 1.

The method according to the invention for the determination and/or removal of a substance in or from a sample by reacting said substance with complexing agents is characterized in that said substance has at least two domains which will interact with complexing agents. As the complexing agents, compounds are used having at least two positions which will interact with said at least two domains of the substance to be removed and/or determined. Those at least two positions may be equal or different in structure, especially considering the structure of said at least two domains of the substance to be determined or removed. According to the invention, the complexing agents are added to the sample. After a complex formation time (incubation time), the sample is sorted by the size of the formed complexes. This is done using a separating device sorting by size in which the formed complexes are retained or reversibly fixed and can be detected. Due to the feature of the method according to the invention, that the substance has at least two domains, and the complexing agent has two corresponding positions, which will interact with one another, the reaction of the complexing agent with the substance to be removed and/or determined yields aggregates which may be significantly larger than the corresponding starting compounds, the substance to be removed and/or determined and the complexing agents.

Thus, it becomes possible to separate the complexes from the starting compounds. According to the invention, this is done using the size as a selection criterion.

Therefore, the separating devices according to the invention must be capable of retaining the formed complexes whereas the starting compounds, the substance to be determined and/or removed and the complexing agents, can pass through the separating device.

Preferably, the substance to be determined is an at least divalent antigen. However, the substance to be determined may also be an antibody which will interact with an antigen. In addition, the systems which can alternately serve as the analyte or detector according to the invention include: enzyme/substrate pairs and receptor/mediator pairs. For example, a receptor ligand may be detected by using the corresponding receptor, which may be important, for example, in clinical diagnostics. Preferably, the divalent antigen is reacted with a complexing agent consisting of at least two different monoclonal antibodies or polyclonal antibody mixtures or their respective fragments. If an epitope is manifested at least twice on the antigen, it is sufficient to perform a corresponding reaction with at least one monoclonal antibody. By means of those relationships, the principle of the present invention is further illustrated.

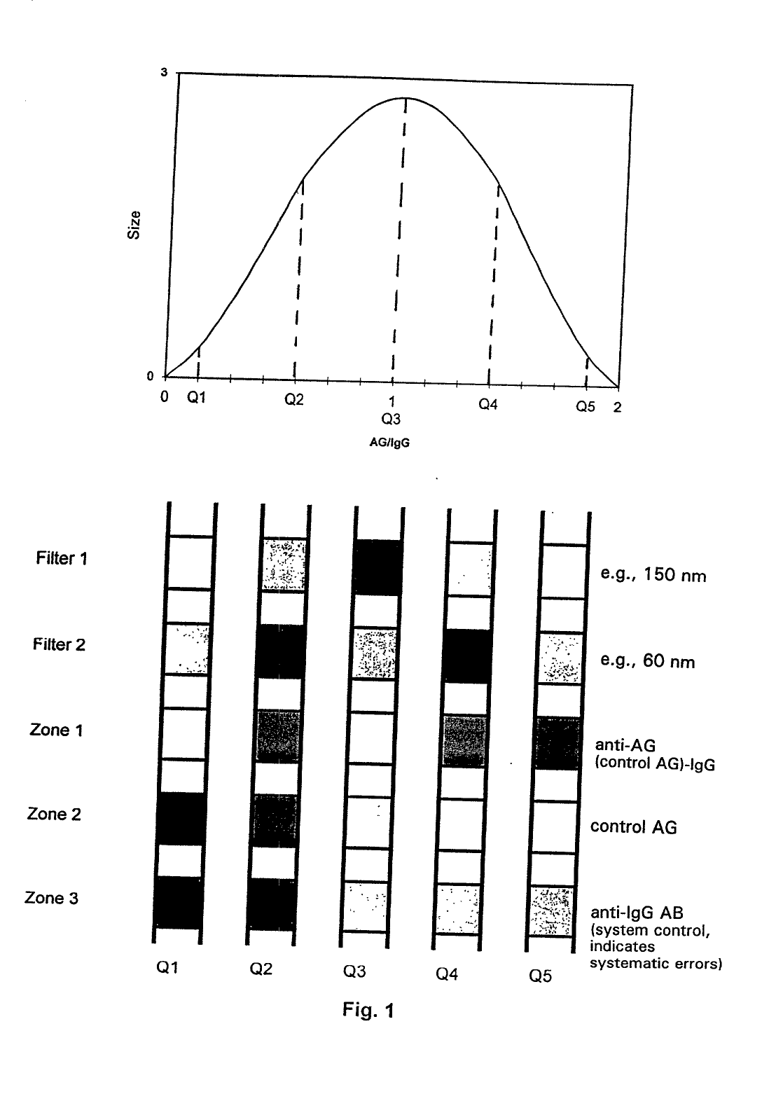

FIG. 1 shows an idealized relation between the size of immune complexes as a function of the quotient Q of the concentration of the antigen, divided by the concentration of an antibody interacting with said antigen, assuming that the affinity is virtually infinite. It can be seen therefrom that the largest complex will form if said at least divalent antigen and said polyclonal antibody or said antibody mix consisting of at least two monoclonal antibodies are present in an approximately stoichiometric ratio. In this case, particularly long-chain immune complexes will form. This situation is known per se and represents the principle of the phenomenon of immunoprecipitation.

If the concentration of the antigen or alternatively that of the antibody is prevailing, correspondingly shorter-chain antigen/antibody complexes will form which have a smaller size accordingly.

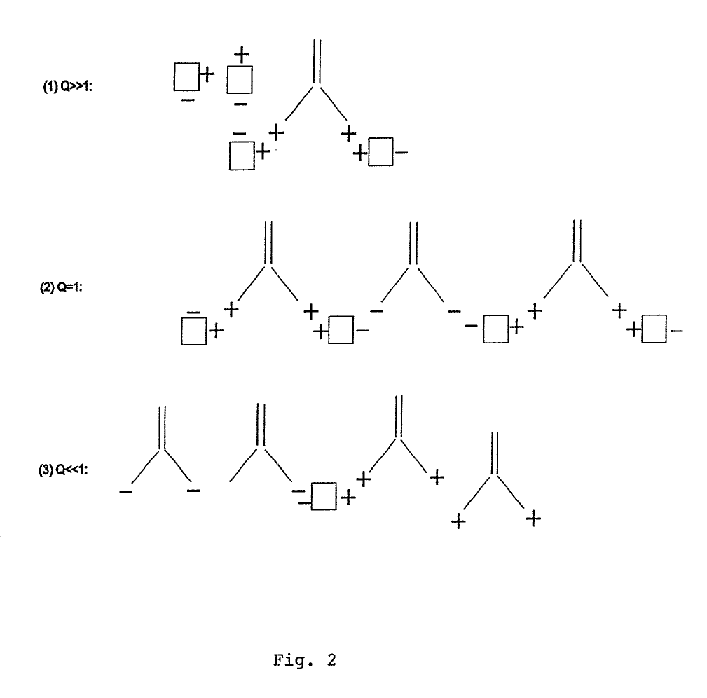

FIG. 1 shows assumed concentration values Q at different positions (Q₁, Q₂, Q₃, Q₄ and Q₅). At these positions, a situation approximately as that shown in FIG. 2 exists. For example, if Q is much larger than 1, it means that the concentration of the antigen is highly prevailing so that virtually all antibody idiotopes can be saturated with one antigen each. Thus, complexes will form from two parts of antigen and one part of antibody. However, if approximately eguimolar ratios between the antigen/antibody starting substances are present, long chains are obtained, typical of immunoprecipitation. In the range of Q<1, i.e. the antibody concentration is relatively high, virtually all antigen molecules will bind two antibodies each so that complex formation with the production of chains does not occur, and complexes of smaller size will form.

Thus, it is possible to establish whether immune complexes are present by separating the immune complexes by their size, followed by detection. Then, it has to be ensured that the separating device sorts by the size of the complexes so that complexes are retained which approximately consist of one antigen and two antibodies or one antibody and two antigens. Thus, if the size of the immune complexes of antigen and antibody or antibody fragment to be expected is known, the separating device can be designed correspondingly to enable it to retard the complexes to be expected. For example, if the concentration of the substance to be determined is on the order of that of the complexing agents employed, a separation by size will surely be possible since the formed complexes at any rate remain in the separating device. This is also the case with more unfavorable ratios of concentrations, i.e., when the antibodies are prevailing or when the antigens are prevailing, since the smaller complexes of the kind described above are also retained by preliminarily selecting the separation performance of the separating device.

According to the invention, the separating device has separating means with a size exclusion threshold of from 20 to 1,000 nm. That is, the separating means in the separating device ensure that complexes with corresponding sizes are retained. As the separating means, there may be used, in particular, submicron filters. Thus, for example, cellulose acetate filter membranes which are commercially available (Millipore, Gelman and Whatman) may be employed. Appropriate membranes or thin films of polycarbonate are also possible. Materials made of hydrophilized PVDF may also be employed.

In a preferred embodiment of the method according to the invention, it is performed using two separating means. The two separating means are distinguished by their different size exclusion thresholds.

If the complex formed in the sample to be examined is first passed through a separating means with larger size exclusion thresholds, those complexes are retarded which form when the concentrations of the components participating in the complex formation reaction are approximately on the order of Q=1. However, if the ratio of concentrations is significantly higher than 1 or significantly smaller than 1, complexes of smaller dimensions will form, as set forth above, so that they can pass the separating means with the relatively large size exclusion thresholds without difficulty. However, they will then be retained in the downstream separating means with the smaller size exclusion threshold.

Any complexes retained in the respective separating means are detected and evaluated depending on where the immune complexes have accumulated. If the complexes formed are found in the separating means with the large size exclusion threshold, it means that the concentration of the antibodies is approximately the same as the concentration of the antigens. In this case, semiquantitative results can already be formulated. However, if the antigen is present either in a large excess or deficiency, it will be found only in the separating means with the small size exclusion threshold. A statement on the ratio of concentrations is not readily possible then because it cannot be seen whether Q is larger or smaller than 1.

Thus, it may be advantageous to connect a control zone with the separating means by which a further differentiation is enabled. Thus, a control zone coated with anti-antigen immunoglobulins can be used to establish whether Q is small or large as compared to 1. Namely, if Q is very much smaller than 1, virtually no more free antigen will be present in the sample so that the control field containing anti-antigen IgG cannot be occupied by free antigen from the sample. Thus, when the control zone is developed with corresponding secondary antibodies or by other detection methods, this control field will remain negative. However, if Q is large as compared to 1, which is equivalent to the statement that a high concentration of antigen is present in the sample, the control field will react to a considerable extent with residual free antigen (the complexing agent was not able to complex all antigen molecules in the sample), so that development of the control field will reveal a correspondingly high extent of complex formation in the control field. This means then that a high concentration of antigen was present in the sample.

There may also be provided a test field in which an authentic antigen is located which is to be detected in the sample. In this case, if Q is smaller than 1, the corresponding test field will indicate a reaction with excess antibodies. Such a control field is kind of complementary to the control field which contains an anti-immunoglobulin antibody.

In addition, it may be advantageous to provide a control zone in which anti-IgG antibodies are located in order to reveal systematic errors when the result is negative.

The detection of the complexes formed is effected in a per se known manner, preferably by secondary reactions, such as detection by means of fluorescence-labeled antibody fragments, specific peptides interacting with parts of the antibodies, etc. In addition to fluorescence-labeled materials, there may be used those labeled with an enzyme and undergoing reactions with corresponding substrates which lead to detectable substances, for example, dyes (horse radish peroxidase labeled substrates). Radiolabeled substrates may also be employed, however.

The method according to the invention has the following advantages, in particular, as compared to the immune quick tests available to date:

coating, washing and staining steps can be included in one single step; special washing methods can be dispensed with;

IgG may be used as a gold (Ag, Se) complex which results in exact requirements with respect to the size of the starting reactants;

the detection limit may be considerably lowered as compared to other quick tests by using enzyme conjugation; detection limits of about 1 pg are possible;

the quick test can be interpreted at least semiquantitatively which is essential to quick tests for establishing physiologically relevant compounds, for example, troponin diagnostics in myocardial infarction quick tests.

The method according to the invention can be realized in a particularly advantageous manner in the device according to the invention having the features of claim 9. The subsequent sub-claims 10 to 14 relate to preferred embodiments of the device according to the invention.

The device according to the invention has a sample applying device, an outlet and a separating device. In the separating device, at least one separating means is provided which is capable of retaining a complex of the substance to be determined and/or removed. The separating means preferably has a size exclusion threshold of from 20 to 1,000 nm.

If the method according to the invention is to be used merely to establish the presence of a substance to be determined and/or removed, it is sufficient if one separating means is present. In more precise evaluations, especially semiquantitative evaluations, it may be advantageous if at least two separating means with different size exclusion thresholds are present. Preferred are a first separation means with a size exclusion threshold of from 150 to 300 nm and a second separation means with a size exclusion threshold of from 80 to 160 nm. If a third separating means is to be provided, a size exclusion threshold of from 20 to 80 nm will be recommendable. The separating device is preferably located in the outlet of the device according to the invention.

The sample to be examined is placed in the applying device, and then the complexing agent or agents are added. Optionally, the complexing agent may already be present in the applying device. It is particularly preferred that the complexing agent may be disposed is a separate space in the applying device which is secluded, for example, by a membrane. After applying the sample, the membrane can be disrupted, for example, by a pulse of pressure, so that complexing agent and sample to be examined can be intimately mixed.

After the incubation time, the mixture is then transferred to the separating device. In a preferred embodiment of the invention, the separating device is provided in the outlet of the sample applying device. In this case, the outlet is optionally closed by a membrane initially. After incubation of the sample, a membrane closing the outlet of the separating device according to the invention can be disrupted so that the mixture of complexing agents, complex and sample to be examined enters the outlet, passes the separating means provided in the separating device, and then emerges from the outlet.

For accelerating the passage of the sample through the device according to the invention, a means may be provided on the side of the outlet facing away from the sample applying device, for example, an absorbent felt, which exerts a force through the outlet by the action of capillar forces so that the sample to be examined entirely passes the outlet.

According to the invention, a flow of the sample to be examined through the device according to the invention may also be produced by applying a pressure on the side of sample application or a reduced pressure on the side opposite to the side of sample application of the device according to the invention. In particular, pumps and centrifuges may be employed.

In order to check whether interferences have negatively affected the reaction or in order to obtain other information, it may be advantageous to provide corresponding test fields preferably in the outlet downstream of the separating device (as seen in the direction of sample flow through the outlet). Thus, a first control field which contains the substance to be determined and/or removed, and/or a second control field which contains a substance which will interact with the complexing agent or agents may be provided.

The method according to the invention may be employed, in particular, in the analysis of room air, food monitoring, drug screening, mold fungus analysis, laboratory medicine, and environmental analysis.

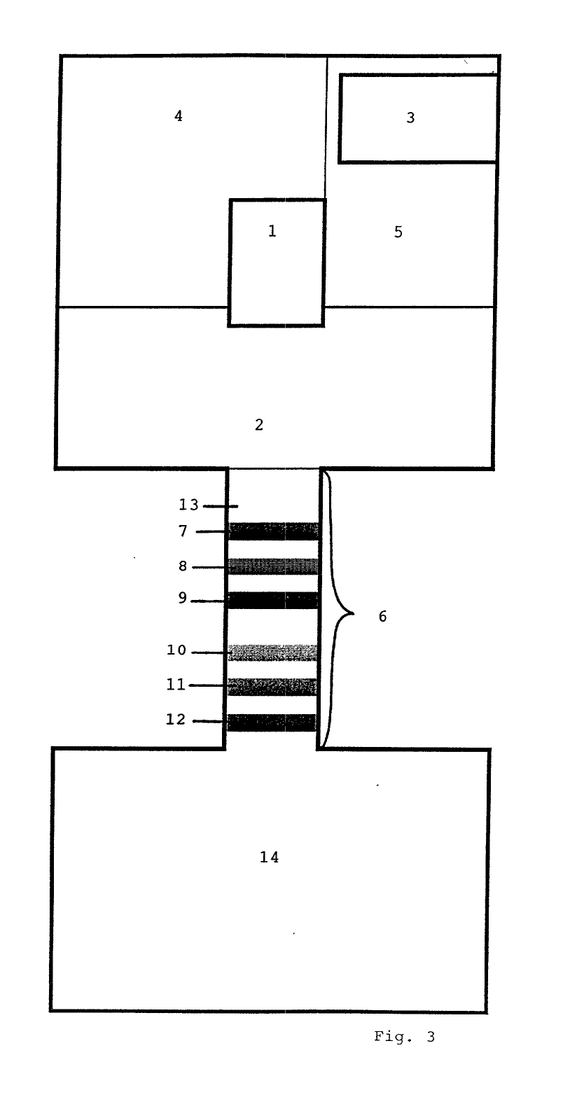

The use of the device according to the invention as an immunodiagnostic agent will be explained in more detail in the following (FIG. 3).

The sample to be examined with the substance to be removed and/or to be determined is filled in sample receptor device 1 through an inlet orifice, and passes through a filter, especially a paper filter, into reaction compartment 2. In reaction compartment 2, it is contacted with lyophilized gold-labeled enzyme-IgG. The device is open to the bottom so that a free distribution throughout reaction compartment 2 may occur.

By pressing on press point 3, a mechanical means such as a slide is actuated, or a membrane is pierced, so that washing solution 4 and staining solution 5 now enter in succession into reaction compartment 2.

Within a short period of time, the reaction mixture with the antibody antigen complexes is passed to separating device 6 and separated there on the first separating means 7, second separating means 8 or third separating means 9 according to size, or subsequently according to affinity. The first separating means 7 has a size exclusion threshold of, e.g., 200 nm, the second separating means 8 has one of, e.g., 120 nm, and the third separating means 9 has one of, e.g., 60 nm. If colored solutions are employed, subsequent washing steps may be recommendable.

Detection is effected by a staining solution which is adjusted such that a constant and small amount of dye remains in the analyzing compartment after the solutions have passed through, so that overstaining is not possible, and an exactly predetermined time is not necessary. The staining solution is, in particular, a precipitating dye and is generated by an enzyme reaction, e.g., with immune reactions, by BCIP/NPT or DAB.

Control fields 10, 11, 12 are provided downstream from separating means 7, 8, 9 and also within outlet 13 of the device. Control field 10 may contain, for example, the antigen to be examined, control field 11 may contain an anti-antigen IgG while control field 12 may contain an anti-IgG antibody.

Outlet 13 is in communication with an absorbent felt 14 which takes up the sample through capillar forces and, together with gravity, ensures that the sample passes through the device according to the invention.

1. A method for the determination and/or removal of a substance in or from a sample by reacting said substance with complexing agents, characterized in that
said substance has at least two domains which will interact with complexing agents;
complexing agents are used which have at least two positions which will interact with said at least two domains;
the complexing agents are added to the sample, and after a complex formation time, the sample is sorted by the size of the formed complexes by means of a separating device sorting by size in which the formed complexes are retained and can be detected.

2. The method according to claim 1, characterized in that the substance to be determined is an at least divalent antigen, divalent enzyme/substrate system, or divalent receptor mediator system.

3. The method according to claim 1 and/or 2, characterized in that said complexing agents are at least two different monoclonal antibodies or antibody fragments which respectively have different idiotopes against said at least two domains of the substance to be determined, or polyclonal antibodies or fragments.

4. The method according to at least one of claims 1 to 3, characterized in that said separating device sorting by size of the formed complexes has separating means which have a size exclusion threshold of from 20 to 1,000 nm.

5. The method according to at least one of claims 1 to 4, characterized in that the substance to be determined is an antigen with at least two epitopes.

6. The method according to at least one of claims 1 to 5, characterized in that at least two separating means having different size exclusion thresholds are employed.

7. The method according to at least one of claims 1 to 6, characterized in that separating means are submicron filters.

8. The method according to at least one of claims 1 to 7, characterized in that said detection of the formed complexes is effected through secondary reactions.

9. A device for performing the method according to at least one of claims 1 to 8, having a sample receptor device (1), an outlet (14) and a separating device (6), characterized in that said separating device (6) has at least one separating means (7) which is capable of retaining a complex of the substance to be determined and/or removed.

10. The device according to claim 9, characterized in that said separating means (7) has a size exclusion threshold of from 20 to 1,000 nm.

11. The device according to any of claims 9 and/or 10, characterized in that at least two separating means (7, 8) having different size exclusion thresholds are provided.

12. The device according to claim 11, characterized in that a first separating means (7) with a size exclusion threshold of from 150 to 300 nm, a second separating means (8) with a size exclusion threshold of from 80 to 160 nm, and optionally a third separating means (9) with a size exclusion threshold of from 20 to 80 nm are provided.

13. The device according to at least one of claims 9 to 12, characterized in that there is provided a first control field (10) which contains the substance to be determined and/or removed, and/or a second control field (11) which contains a substance which will interact with said complexing agent(s).

14. The device according to at least one of claims 9 to 13, characterized in that said separating device (6), said first and/or second control field (10, 11) are provided within said outlet (14).
